(19) United States
(12) Patent Application Publication
(10) Pub. No.: US 2021/0330851 A1
(43) Pub. Date: Oct. 28, 2021

(54) FACE MASK WITH ENHANCED UV-C STERILIZATION FLOW PATH AND LOW RESISTANCE TO INHALATION

(71) Applicant: Adam Warwick Bell, San Francisco, CA (US)

(72) Inventor: Adam Warwick Bell, San Francisco, CA (US)

(21) Appl. No.: 16/860,888

(22) Filed: Apr. 28, 2020

(51) Int. Cl. A61L 9/20; A62B 18/02

(52) U.S. Cl. CPC A61L 9/20; A61L 2209/12; A62B 18/025

(57) ABSTRACT

A face mask designed for easy breathability, incorporating a UV-C light source, designed to have a long air flow pathway and/or a long dwell time, designed to protect the wearer against disease-causing biological agents.

### RELATIONSHIP TO OTHER APPLICATIONS

None

### FIELD OF THE INVENTION

The present invention relates to a face mask that kills airborne pathogens, incorporating a UV-C light source, and maximizing the air flow pathway, and designed to provide easy breathing through the mask.

### BACKGROUND

Many human illnesses are transmitted from one individual to another by aerosols or fomites.

The 1918 “Spanish” influenza pandemic was caused by an H1N1 influenza virus. It infected 500 million people around the world (about 27% of the then world population) and is estimated to have killed about 60 million people. Annual flu epidemics result in a yearly average of about 65,000 deaths globally.

Severe acute respiratory syndrome (SARS) was caused by the SARS coronavirus (SARS-CoV), and between November 2002 and July 2003, an outbreak in China caused about 8,098 cases and 774 deaths (9.6% fatality rate).

COVID-19 first emerged in Wuhan, Hubei, China and is the cause of the present 2019-20 coronavirus pandemic. Its effect and severity is yet to be determined. The novel coronavirus is a positive-sense single-stranded RNV virus, same as the SARS and MERS virus. Researches on SARS found that this kind of virus is sensitive to heat radiation and UVC light and can be diminished when exposure to UVC irradiation stronger than 90 μW/cm2. Thus, in theory, UV light would be able to destroy the novel coronavirus (2019-nCov).

The person-to-person transmission of influenza virus, especially in the event of a pandemic caused by a highly virulent strain of influenza, such as H5N1 avian influenza, is of great concern due to widespread mortality and morbidity. Because airborne transmission is key, public health interventions should focus on preventing or interrupting this process. Scientists have known for decades that broad-spectrum UVC light, which has a wavelength of between 200 to 400 nanometers (nm), is highly effective at killing bacteria and viruses by destroying the molecular bonds that hold their DNA together. This conventional UV light is routinely used to decontaminate surgical equipment. Influenza A virus (H1N1, PR-8) aerosols that are exposed to UV-C light using doses ranging from 4 to 12 J/m2 effectively kill the virus, and it has been shown that far-UVC efficiently inactivates airborne aerosolized viruses, with a very low dose of 2 mJ/cm2 of 222-nm light inactivating >95% of aerosolized H1N1 influenza virus.

Respirators (N-95 and N-100; both commercially available) are masks designed to shield the wearer from inhalational hazards, as opposed to surgical masks, which are designed to protect others from contaminants generated by the wearer. Both are difficult to use effectively because of movement and entrance of air around the sides of the mask.

One major problem with masks and respirators is that in order to filter out very small particles, the air has to pass through dense filters with considerable air resistance. This makes them more uncomfortable to wear and difficult to use, meaning that compliance is low. Also, because the air has to pass through an area of high resistance, the air is likely to try to find the path of least resistance and to find an unfiltered pathway, if such exists, letting in unfiltered air from the outside. This happens even if the filter has a large surface area. The invention overcomes this problem by providing sterilization of air in a pathway, whereby such air can enter through a low resistance pathway into a flow path or chamber where it is sterilized. Thus the air does not have such a tendency to travel through a pathway of least resistance, as the resistance of the desired pathway is low.

UV light is electromagnetic radiation with a wavelength shorter than that of visible light. UV light is categorized as consisting of 3 wavelength bands, each of which has different properties. Ninety-nine percent of the UV light that reaches the earth's surface is UV-A (400-320 nm). UV-B (320-290 nm) is responsible for skin tanning and sunburn and with long exposure may cause skin malignancies and cataracts. UV-C has the highest energy of the UV light bands. The primary germicidal range for UV light is 260 to about 280 nm, which is within the UV-C band. The theory behind use of UV light to disinfect air is that respiratory infections may be spread by suspended aerosols.

Broad-spectrum UV-C light, which has a wavelength of between 200 nm to 400 nm, is effective at killing bacteria and viruses. The band of light with a wavelength between 200 nm and 280 nm is called the UV-C band, and is used in the present invention.

Low-pressure mercury lamps emit a single output at 253.7 nm; the total output power of the lamp is equivalent to the output power in the UVC range, albeit at a non-optimal wavelength. Medium-pressure mercury lamps emit a broader range of wavelengths that include the germicidal range, but also unnecessary wavelengths for disinfection purposes, but only about 20-30% of the light is emitted in the UVC range. On the other hand, the continuous spectral response of UVC LEDs is predominantly within the desired UVC range, which allows for a more efficient system.

At germicidal UV wavelengths, adjacent thymine bases dimerize, rendering viral or bacterial DNA and RNA incapable of replication. Germicidal lamps typically emit UV-C at 16000 μW s/cm2. See Weiss et al, Am J Public Health. 2007 April; 97: S32-S37 and International Ultraviolet Association. UV-FAQs Frequently asked questions about UV. Available at: http://www.iuva.org/public/faqs.php. Aerosol Susceptibility of Influenza Virus to UV-C Light has been shown to be an efficient process. See Applied and Environmental Microbiology p. 1666-1669 March 2012 Volume 78 Number 6. Air disinfection via upper-room 254-nm germicidal UV (UV-C) light in public buildings may be able to reduce influenza transmission via the airborne route. Susceptibility of influenza A virus (H1N1, PR-8) aerosols to UV-C has been demonstrated using UV-C doses ranging from 2 to 12 J/m2.

Far-UVC at 222 nm inactivates more than 95% of airborne aerosolized H1N1 influenza viruses at a low dose of 2 mJ/cm2. Because light at wavelengths from 207 to 222 nm are completely absorbed by the dead outer layer of skin and by the outer tear layer of the eye, these wavelengths are safe for humans (unlike the commonly used 254 nm germicidal wavelength that can cause skin cancers, including deadly melanoma). See publication titled “UV Sterilization: Far-UVC light kills airborne flu viruses without danger to humans” John Wallace, Apr. 1, 2018 (https://www.laserfocusworld.com/lasers-sources/article/16555364/uv-sterilization-faruvc-light-kills-airborne-flu-viruses-without-danger-to-humans).

A table of effective UV Irradiation Dosage is provided by American Air & Water, Inc. and can be found at www.americanairandwater.com/uv-facts/uv-dosage.htm. Tests conducted by Light Sources Inc. and verified by American Ultraviolet Company—Lebanon, IN revealed that the American-Lights lamp produces 800 μW/cm² at 1 inch with 534FPM air flow at 55 F. UV dose=UV intensity×time in seconds. To compute time needed to inactivate germs in the following chart at 1′ distance divide the UV dose by 800. Example: for 90% kill factor of influenza: 3,400 divided by 800=4.25 seconds. The following are incident energies of germicidal ultraviolet radiation at 253.7 nanometers wavelength necessary to inhibit colony formation in microorganisms (90%) and for 2-log reduction (99%):

| | Energy Dosage of Ultraviolet | |
|---|---|---|
| | radiation (UV dose) in μWs/cm² | |
| Organism | needed for kill factor | |
| Virus | 90% | 99% |
| Bacteriopfage - E. Coli | 2,600 | 6,600 |
| Infectious Hepatitis | 5,800 | 8,000 |
| Influenza | 3,400 | 6,600 |
| Poliovirus - Poliomyelitis | 3,150 | 6,600 |
| Tobacco mosaic | 240,000 | 440,000 |

### BRIEF DESCRIPTION

The invention encompasses a face-mask designed to protect the wearer against disease-causing biological agents. Specifically, a face mask designed to kill viruses and bacteria using sterilization affected by UV-C light, and UV light with a wavelength of 200-280 nm, preferably 250-280 nm, appears to be particularly effective at killing microbes.

### BRIEF DESCRIPTION OF THE FIGURES

FIG. 1 shows the mask having a linear/parallel design so that the flow path is made up of a number of approximately parallel elongated chambers. 1=outside surface of mask; 2=interior surface of mask; 3=flow path; 4=flow path baffles or walls separating portions of the elongated chambers; 5=air inlet which optionally includes a valve; 6=path of flow of the air into the mask; 7=unit comprising UV-C emitting light source; 8=one or more diodes providing UV-C radiation; 9=cell/battery; 10=switch; 11=conductor; 12=interior chamber of mask.

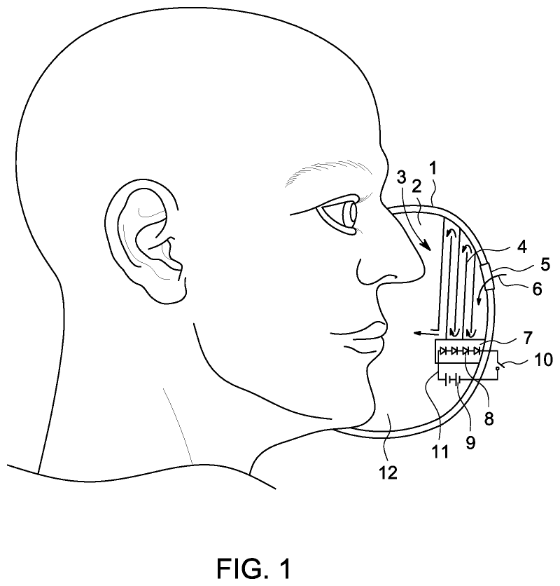

FIG. 2 shows the mask having a radial design. 13=light emitting diode; 14=baffles separating portions of the flow path; 15=path of flow of air entering; 16=path of flow of air exiting; 17=air inlet; 18=air vent/exit.

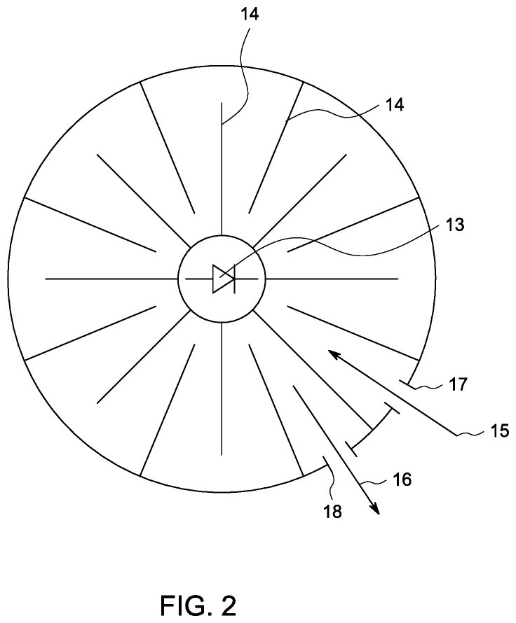

FIG. 3 shows the mask having a dwell chamber (“sterilization chamber”) design. 19=inhalation chamber proximal to the user, covering the user's nose and mouth; 20=sterilizing anti-chamber (“dwell chamber”) distal to the user; 21=interior of inhalation chamber; 22=interior of dwell chamber which may be coated with reflective material; 23=air inlet which may comprise a valve; 24=valve between chambers; 25=conductor; 26=switch; 27=cell/battery; 28=LED.

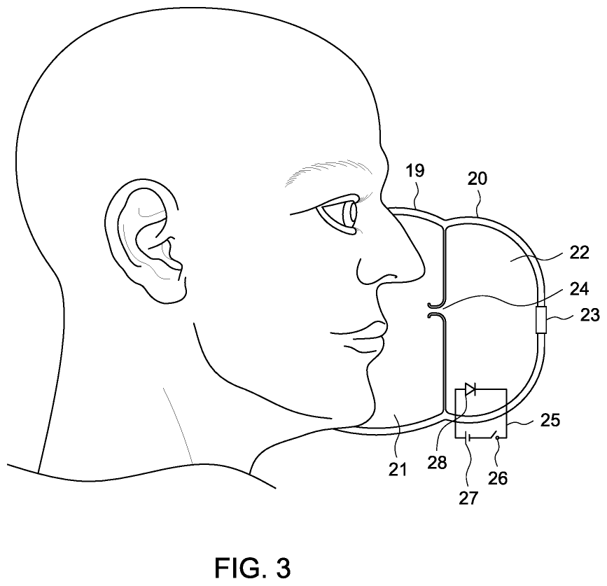

### DETAILED DESCRIPTION

UV light has been known for many years to kill microorganisms, and has been generally used for sterilization of surfaces and water. Broad-spectrum UV-C light, which has a wavelength of between 200 to 400 nanometers, is highly effective at killing bacteria and viruses by destroying the molecular bonds that hold their DNA together. This conventional UV light is routinely used to decontaminate surgical equipment. More recently UVC has been identified as an effective type of electromagnetic radiation to eliminate viruses and bacteria while minimizing harm to a human user. Specifically, UV light with a wavelength of 200-280 nm appears to be most effective as a germicide. UV-C doses ranging from 1 to 12 J/m2 prove to be very effective at killing viruses and bacteria. Far-UV-C may have enhanced benefits because it has a very limited range and cannot penetrate through the outer dead-cell layer of human skin or the tear layer in the eye, therefore it does not pose a hazard to human health. However, viruses and bacteria are easily killed by far-UV-C light.

The present invention encompasses a face-mask designed to protect the wearer against disease-causing biological agents.

Specifically, the face mask of the invention is designed to kill viruses and bacteria using sterilization affected by UV-C light. UV light with a wavelength of 200-280 nm appears to be particularly effective at killing microbes.

Light with a wavelength between 200 nm and 280 nm (UV-C band) is used in the present invention to kill bacteria and viruses in a volume of air. In one embodiment, 1-24 mJ/cm2 of light at a wavelength of 210-230 nm is used. This will inactivate >90% of aerosolized H1N1 influenza virus or similar enveloped viruses. UV light with a wavelength of 200-280 nm is generally used herein, and between 200 and 240 nm is thought to be ideal and is preferably used herein.

The present invention includes a compact, low wattage UV-C light source such as a light emitting diode (LED) which is adapted to illuminate an air flow pathway. Alternatively a UV LASER source may be used.

Air flows through an airflow pathway in which it is exposed to UV-C. The airflow pathway has a first end or ends (entrance or intake), through which atmospheric air enters, and a second end (exit) through which the air exits towards to user, to be inhaled by the user. The entrance is positioned on the outside of the mask in contact with atmospheric air, and the exit is positioned within the mask.

The air flow pathway is designed to maximize the residence time (sometimes referred to as ‘dwell time’) of pathogen particles within the pathway so that said particles receive a significant exposure (i.e. a significant time and overall exposure) to UV-C energy, sufficient to kill or inactivate or attenuate a significant proportion of the organisms. The pathogens are directed through an elongated pathway which may be convoluted or folded back on itself or coiled in such a way that the particles are exposed to UV-C during nearly all the time they travel through the pathway.

The flow pathway may have various geometries, and may be elongated, repeatedly folded in a parallel manner, radial, or coiled (all described below), but is at least 30 cm in total length. The UVC light is positioned within the device in such a way as to directly illuminate (i.e., in a straight line) at least 90% of the interior of the flow pathway. It may directly illuminate >90%, >95%, >98% or >99% the interior of the flow pathway. In most embodiments, the pathway will turn or flex at various points which will result in certain areas not receiving direct UVC illumination. But in the coiled embodiment, the illumination may be substantially 100%.

In the “Sterilization chamber” design (described below), the definition “flow pathway” is broadened to include not an elongated pathway, but a chamber. The air still flows through this pathway (chamber) from the outside atmosphere, through the flow pathway to the nose and mouth of the user, but rather than being an elongated pathway with a length many times greater than it's diameter, width or height, the aspect ratio is much lower with the flow path being more of a box, bag, sphere or semi-sphere shape, so that the flow pathway is in fact a “dwell chamber” in which a certain volume of air is retained between breadths, and is subjected to UVC while it dwells within the sterilization chamber. In this embodiment, the illumination may be >75%, >90%, >95% or >99%.

A “significant exposure” can be defined as an exposure that can on average, or on a single application during the time the pathogens are within the pathway (the “residence time”), kill or inactivate at least 25% or 35% or 50% or 75% or 90% or 95% or 99%. Any range that included these amounts is also implied, for example from x % to y % or at least x % or between x % and y % or no more than x %, where x and y could be any number. 2 mJ/cm2 of light at a wavelength of 222 nm has been shown to inactivate >95% of aerosolized H1N1 influenza virus.

A significant exposure can also be defined as an exposure to a certain amount of total energy. Or it may be defined as a certain amount of energy per unit time or per unit area or unit volume. The UV dose can be expressed as UV intensity×time in seconds. For example, in this invention, significant exposure can be 1-20 mJ/cm2 of UV light with a wavelength of 200-280 nm. More specifically it can be 2-12 mJ/cm2. Or in other embodiments can be 2, 4, 6, 8, 10, 12, 14, 16, 20 or 24 mJ/cm2. UV light with a wavelength of 200-280 nm is generally used herein, and between 200 and 240 nm is thought to be ideal. Alternatively, a UV dose may be expressed in μW/cm². In one example, 2-12 mJ/cm2 of light at a wavelength of 222 nm has been shown to inactivate >95% of aerosolized H1N1 influenza virus.

When we discuss energy exposure using joules per unit area, the unit area may be calculated as the cross-sectional area of a pathway (tube, box etc.), drawn “in the air” to slice through the length of the tube orthogonal to its length, and it does not necessarily imply (but may include) a physical surface.

The stated exemplary amounts do not limit the invention. Any range that includes the stated amounts is also implied, for example from x to y or at least x or between x and y or no more than x, where x and y could be any number with appropriate units of, for example, mJ/cm2 or W/cm².

When “killing” of a pathogen or organism such as a bacteria or virus is mentioned, this includes and implies inactivation, attenuation, killing, denaturing, or otherwise affecting the pathogen so that infectivity or pathology is reduced. Organisms do not have to be killed in order to be changed in a way that reduces the significance or severity or amount of disease caused, and all these embodiments are explicitly included.

The mask is designed in the mode of many conventional masks to fit around the mouth and nose or a person or animal. The mask may be airtight so that all air travels through the UV-C exposed pathway; or substantially airtight so that substantially all air travels through the UV-C exposed pathway, or may not be airtight such that some air can enter without passing through the UV-C exposed pathway. Substantially all air means at least 50% of air, or preferably at least 75% or air, or more preferably, at least 85%, 90% or 95% or 99% of air that is inhaled by the user travels through the UV-C exposed pathway.

Pathway designs. The pathway is generally elongated or convoluted and in one embodiment, the flow pathway folds back and forth in such a way that the volume of air in the pathway is nearly always directly exposed to the UV-C light from the light source. Most exposure is direct, within a straight “line of sight” of the light source, and not by virtue of reflection.

The parallel pathway design. In the “parallel pathway design” the pathway comprises a plurality of substantially straight patent hollow paths (tubes, corridors, tunnels—we will refer to them as “paths”) each defined by walls, connected in such a way that the length of the path extends in a straight line from the light source. One path may be connected to the next at any location, generally the end of one path is connected to the beginning of the next to produce a continuous flow path. Air is drawn from one corridor to another, always travelling through the corridors either towards or away from the light source, but almost always within line of sight of the light source. The plurality of paths are more or less parallel to each other in the “Parallel Pathway” design, and one or more UV-C light sources is/are positioned such that they resides at the end (one or both ends) of the paths, such that air that flows through the paths is almost always exposed in line of sight to light emitted from the light source.

The radial pathway design. In another architecture, (the “radial pathway design”), the plurality of paths radiates outward like spokes from the central UV-C light source, either in a 2-dimensional arrangement like a cartwheel, or a 3-dimensional arrangement like a dandelion.

The coil pathway design. In a further embodiment (the “Coil Pathway” design), the path is in the shape of a coil and the UV-C source(s) is/are positioned flat against the coil, so that the air travels through the coil, continuously exposed to UV-C.

In any of the above embodiments, any volume or pathway or chamber into which UV light is shone, such as the flow pathway (or the sterilization anti-chamber in another design), may be coated with a reflective material. Reflection of UV-C increases killing power. The interior of the anti-chamber can be made or/lined/coated with a reflective coating. A common material used in commercial UV disinfection systems is stainless steel. While this surface is highly resistive to microbial growth, it only has 20-28% reflectance of UV light. Flow cells that contain e-PTFE (expanded PolyTetraFluor Ethylen) provide more than 95% reflectance (as shown in the table above) of the UVC light—more than three times effective. Thus e-PTFE (expanded PolyTetraFluor Ethylen) may be used to line the flow pathway or any chamber of the invention. Other shiny materials may be used including aluminum (such as aluminum foil), mirrored glass, titanium dioxide etc. Additionally, any material may be used that has a greater than 90% reflectivity for UVC, or any other suitable material may be used, such as any material mentioned in U.S. Pat. No. 965,717, which teaches a UVC reflective coating; U.S. Pat. Nos. 3,956,201, and 5,892,621 which teach fluorinated polymers and air void morphologies to scatter a broad spectrum of light; U.S. Pat. No. 7,511,281 teaches an Ultraviolet Germicidal Irradiation (UVGI) sterilization process; U.S. Pat. No. 3,300,325 teaches air-void filled coating compositions for UVA and UVB reflecting paint. All these are expressly incorporated by reference.

The sterilizing chamber (“anti-chamber”) design. An advanced embodiment (the “sterilizing chamber” embodiment) produces a longer dwell time during which organisms are exposed to UVC. It employs a separate inhalation chamber proximal to the user, covering the nose and mouth, and a sterilizing chamber (and anti-chamber) distal to the user, and a valve located between these two chambers. Upon taking a first breath, a certain volume of air (equivalent to the volume of a breath) enters a sterilizing chamber but does not get inhaled due to the natural lung capacity of a person, so that this volume of air is retained in the chamber until a second breath is taken, and then passes through the valve and is inhaled. The inhalation chamber is in proximity to the user's mouth and nose, and is separated from the sterilization chamber by a valve. The valve opens when negative pressure (a vacuum) is applied by the user breathing in. Ideally, the sterilization chamber should have a volume similar to the tidal breath volume, which on average in a healthy man or woman is 0.5 litres. Structurally, the sterilizing chamber may be located within or outside the main mask structure.

The sterilization anti-chamber has located therein, one or more UV-C light sources. The UV-C light source may illuminate the interior volume of the sterilization chamber to kill pathogens. The purpose of the sterilization chamber is to increase dwell time such that the air retained therein is present for between about 1 and 7 seconds, more typically between 2 and 6 seconds, depending on the rate of the user taking a breath. The typical respiratory rate for a healthy adult human at rest is 12-18 breaths per minute. Respiratory rhythm is about 2 seconds for an inhalation, and 3 seconds for exhalation, with an average rate at 12 breaths per minute. This means that the dwell time is the time between breaths, and is about 5 seconds for the embodiment using the sterilizing chamber.

The sterilizing anti-chamber may be a single chamber without internal pathways or baffles, having mounted therein a UV-C source. Or it may include a system of pathways as described herein to further increase path length of air travelling from the exterior to the user, and therefore increase the exposure time or energy to which the pathogens are subjected.

In the sterilization anti-chamber design, the definition “flow pathway” is broadened to include not an elongated pathway, but a chamber (a “dwell chamber” or “sterilization chamber”). Thus in the claimed invention, the flow pathway encompasses the dwell chamber, unless otherwise limited. The air still flows through this pathway (chamber) from the outside atmosphere, through the flow pathway to the nose and mouth of the user, but rather than being an elongated pathway with a length many times greater than it's diameter, width or height, the aspect ratio is much lower with the flow path being more of a box, bag, sphere or semi-sphere shape, so that the flow pathway is in fact a “dwell chamber” in which a certain volume of air is retained between breadths, and is subjected to UVC while it dwells within the sterilization chamber. In this embodiment, the illumination may be >75%, >90%, >95% or >99%.

In the sterilization anti-chamber design, reflection of UV-C increased killing power. Preferably, e-PTFE (expanded PolyTetraFluor Ethylen) may be used to line the chamber. Other shiny materials may be used including aluminum (such as aluminum foil), mirrored glass, titanium dioxide etc. Additionally, any material may be used that has a greater than 90% reflectivity for UVC, or any other suitable material may be used, such as any material mentioned in U.S. Pat. No. 965,717, which teaches a UVC reflective coating; U.S. Pat. Nos. 3,956,201, and 5,892,621 which teach fluorinated polymers and air void morphologies to scatter a broad spectrum of light; U.S. Pat. No. 7,511,281 teaches an Ultraviolet Germicidal Irradiation (UVGI) sterilization process; U.S. Pat. No. 3,300,325 teaches air-void filled coating compositions for UVA and UVB reflecting paint. All these are expressly incorporated by reference.

The other embodiments that only expose air to UV-C while it is being inhaled, and is moving through the sterilization pathway, have a shorter dwell time, of about 1-2 seconds. However this is perfectly sufficient to reduce pathogen numbers if UV-C intensity is sufficient.

It should be remembered that as light is radiated from a point source, the power per unit area (intensity) is inversely proportional to the square of the distance from the light source. Therefore it is advantageous to keep the pathogens within a path that is as close as possible to the light source.

In a tubular path with the source at one end, assuming light does not reflect from the internal sides of the tube, light intensity is much higher at the end proximal to the source, and lower at the end distal from the source. This may be perfectly sufficient for the invention, especially if the average power exposure in the tube (i.e. the power exposure half way down the tube, is equal to, or more than 2 mJ/cm2, for example 2-12 mJ/cm2.

In some embodiments the path (and therefore the pathogens to be killed) is kept close to the light source, maintaining high intensity. Pathways may be flattened so that the aspect ratio of width to height is very high (e.g., 1:5, 1:7, 1:10, 1:15, 1:20 and 1:30) and any pathogens therein are exposed to a maximum amount of radiation. Such a configuration may be linear or circular or in the form of a spiral pathway (the “Spiral Pathway design”).

Pathway length varies according to the specific design. For the “Parallel Pathway design”, the total pathway length is between 6 and 100 cm long. Each individual straight pathway is about 4-6 cm long. These pathways may be arranged side by side approximately parallel to each other, and flowably connected so that a gas may pass through the entire pathway, constantly subjected to UV-C. For the “radial pathway design” in which the pathways radiate out from a central UV-C source, the total pathway length may be between 60 and 500 cm long. For the “Spiral pathway design” the total pathway length is between 50 and 200 cm long. The above relate to specific designs, but are not meant to limit the invention to pathways of specific lengths, provided that the principles of the invention remain the same.

Killing capacity. Killing depends on the total amount of energy to which a pathogen is exposed. The relevant factors are wavelength, intensity and time. Wavelength has been discussed and is generally between 210 and 280 nm. Intensity has been discussed and is inversely proportional to the square of the distance of the pathogen from the light source. This is a function of mechanical design. Time of exposure is dependent on dwell time which is dependent on path length and flow rate. Path length is designed to increase dwell time.

Dwell time/Exposure time. Dwell time should ideally be from 1 second to 10 seconds. But of course the greater the intensity of light impinging upon the pathogen, the less time is required to kill a pathogen. In eth present invention, the flow rate depends upon the length of the flow path, the cross-sectional area of the flow path, and the strength and rate of inhalation of the user. For an average user, breathing 2-15 times per minute, with an inhalation time of 1-3 seconds the dwell time during which a pathogen is exposed to UV-C is between 1 and 3 seconds, more typically, at rest, 1-2 seconds. For the embodiment using the sterilizing chamber, the dwell time is 4-7 seconds, usually about 5 seconds. As discussed, Dwell time is about 5 seconds for the embodiment using the sterilizing chamber. Dwell time for embodiments that only expose air to UV-C while it is being inhaled, and is moving through the sterilization pathway, is about 1-2 seconds.

Breath volume. Breath volume may be important in calculating the efficiency of sterilization, and the volume needed by the invention to provide maximal UVC exposure. The tidal volume is the volume of air that is inhaled or exhaled in a single such breath. Tidal volume (TV) in a healthy man or woman is 0.5 litres.

Type of light source. The light source is an LED producing UV-C with a wavelength of germicidal range of between 200 and 300 nm, typically between 260 nm to 275 nm. This may be, for example, a 4 W, 6V LED, sourced from Leadfar corporation in China. This claims to have a killing efficiency of >98% with a 20 second exposure of a surface. The “Ceramic SMD 3535 Package” UV-C LED from Unilumen in China has a peak wavelength of 265 or 275 at 40 mA, a power of 2.0-3.0 mW. Other Unilumen LED sources can run at 100 mA at 8-12 Watts and 6-8 Volts, claiming to “kill 99.99% of harmful bacteria in seconds”. The lifespan of these LEDs is approximately 10000 hours. The “Kinreen Flashlight UVC” is also available commercially and has a specification of 30 mW, voltage of 110V, UVC 280 nm using three LEDs (Each 10 mW), and claims to kill 99.99% of influenza virus with a 10 second exposure. Another manufacturer is Tao Yuan (https://www.ledwv.com/uv/) which supplies various UVC LEDs, such as UVC-LED 3535 SMD 275 nm Electro-Optical characteristics at 40 mA, UVC LED 3535 275 nm 10-18 mW Electro-Optical characteristics at 100 mA, UVC LED 3535 SMD 275 nm Electro-Optical characteristics at 100 mA, UVC Sterilize Strip 275 nm 60×20 mm, 275 nm Deep UVC LED for water Sterilizer Absolute Maximum Ratings at TA=25° C. Item Value Voltage 12-24V. KARLAN corp., again in the PRC, produces many UVC LEDs having peak wavelength of 260-275 nm and total optical power output of 40 mW, 50 mW or 60 mW (see https://www.klaran.com/).

It can be seen that many UVC LEDs are available with low energy requirements, but which produce sufficient power at appropriate wavelength to kill viruses.

Alternative light sources include a UV-C laser. UV lasers and optical emitters are used in medical markets that require sterilization. Lasers that are developed using ultraviolet under DPSS circuitry contain crystals from Nd:YAG/Nd:YVO4 garnet.

Power of the light source. The power consumption of the light source is that which is practical for portability, i.e., power that can be provided by a portable battery of not more than 500 g, and preferably considerably less. Power consumption of each LED may be from about 0.05 mW to 20 W, or preferably from 1 mW to about 5 W, or more preferably from 1 mW to 100 mW, with the power supply being a battery, such as a Li-ion battery, optionally provided from a device such as a mobile phone, or for example, 4×AAA 1.5V batteries providing 6V DC.

Power (P) in watts (W) is equal to the energy E in joules (J), divided by the time period tin seconds (s), i.e.: P(W)=E(J)/t(s). 1 Joule/second=1 Watt.

The UVC LED emits an output of from about 10 to 100 mW in radiometric power. LED life is inversely proportional to radiometric power. For example a commercial UVC LED operated at 350 mA (a typical specified condition by UV-C LED manufacturers) emits an initial output of 33 mW in radiometric power and shows little degradation over 1000 hours of use. In contrast, when operated at 700 mA, the LED may emit close to 60 mW but degrades quickly under this condition.

The power produced by the light source, and used to sterilize the pathogens may be, at the lower end, about 1 or 2 mJ/cm2/s, which is equivalent to 1 or 2 mW/cm2. However it has been stated that power of as low as 90 μW/cm2 (0.09 mW/cm2) can be effective for killing H1N1. Therefore the invention encompasses output power ranges from 0.09 mW/cm2 to 24 mW/cm2, and all amounts and ranges in between. See Applied Optics, 49, 5276-5283 (2010) http://www.opticsinfobase.org/ao/abstract.cfm?URI=ao-49-28-5276

In some embodiments, significant exposure with effective killing can be provided by 1-20 mJ/cm2 of UV light with a wavelength of 200-280 nm. More specifically it can be 1-12 mJ/cm2. Or in other embodiments can be 2, 4, 6, 8, 10, 12, 14, 16, 20 or 24 mJ/cm2. UV light with a wavelength of 200-280 nm is generally used herein, and between 200 and 240 nm is thought to be ideal. Alternatively, a UV dose may be expressed in μW/cm². In one example, 2-12 mJ/cm2 of light at a wavelength of 222 nm has been shown to inactivate >95% of aerosolized H1N1 influenza virus. 1 mW=1 mJ/s.

A good example of a commercially available UVC LED that can easily be run on an inexpensive 9V battery is LG's Model: LEUVA66H70HF00 “High power 70 mW UVC LED UV Disinfection LED” used for water treatment air purification. It has the following parameters: Forward Current: 500 mA; Forward Voltage: 8.5V; Radiant Flux: 70 mW; Power Dissipation: 4.25 W; Lighting Color(Peak Wavelength): 270-285 nm; and Wavelength: 278 nm.

Another good example of commercial UVC LEDs are those produced by Klaran, such as:

| | | Total Optical Power Output |
|---|---|---|
| Part Number | Peak Wavelength | at 500 mA |
| KL265-50W-SM-WD | 260-270 nm | 80 mW |
| (engineering sample) | | |
| KL265-50V-SM-WD | 260-270 nm | 70 mW |
| KL265-50U-SM-WD | 260-270 nm | 60 mW |

The mask body may be made out of various suitable materials, such as paper cotton or other woven or matted materials, including electrostatic media designed to trap particles. An example of the mask would meet N95 standards, and CDC guidelines for Mycobacterium tuberculosis exposure control, and would have 99% BFE (Bacterial Filtration Efficiency) according to ASTM F2101 and be Fluid resistant according to ASTM F1862.

An important factor of the invention is that the resistance to inhaling air through the apparatus is less than for many conventional masks. One major problem with masks and respirators is that in order to filter out very small particles, the air has to pass through dense filters with considerable air resistance. This makes them more uncomfortable to wear and difficult to use, meaning that compliance is low. Anybody who has tried to use a mask for a long time or to perform exercise while wearing a mask will know how difficult it becomes and how the desire increases to take off the mask. Also, because the air has to pass through an area of high resistance, the air is likely to try to find the path of least resistance and to find an unfiltered pathway, if such exists, letting in unfiltered air from the outside. This happens even if the filter has a large surface area, such as with a common 3M face mask. The present invention overcomes this problem by providing sterilization of air in a pathway, whereby such air can enter through a low resistance pathway into a flow path or chamber where it is sterilized. Thus the user does not feel that inhalation is restricted, and does not have such a desire to remove the mask, and also, air does not have such a tendency to travel through a pathway of least resistance, as the resistance of the desired pathway is low.

Air pressure across a membrane or filter is traditionally measured in units of “inches of water column” (w.c.). The mask material will have an inherent air resistance. For a given surface area, the greater the overall air resistance the harder it is to breathe in through the mask.

The mask of the invention may be rated with a total resistance in use of, for example, 0.01 to 0.5 inches of water column (w.c), for example from 0.01 to 0.25 inches w.c. In a typical example, the pressure drop across the filter medium may be from 0.01 to 0.1 inches w.c., for example 0.01 to 0.05 inches w.c. Preferably the filter pressure drop should not exceed 0.10 inches w.c.

One method for measuring Air Permeability is the ASTM D737-96 Standard Test Method https://csbs.uni.edu/sites/default/files/AirPermeability.pdf. Another is found in the publication Drewno 2014 “Air flow resistance across nonwoven filter fabric covered with microfiber layer used in wood dust separation” Vol. 57, No. 191; http://www.drewno-wood.pl/pobierz-12

Another important factor is the definition of the “air inlet”. The mask of the invention comprises an outer surface and an inner surface and an air inlet penetrating from the outer surface to the inner surface, wherein the air inlet is flowably connected to the flow pathway having a first end a second end. It is very important to note that in certain embodiments, the air inlet is not a restricted tube or path leading into a chamber or flow pathway, but the air inlet may comprise the entire surface of the mask, such as in a conventional mask. Air may be drawn in across the whole or a substantial outer surface of the mask.

In some embodiments the “flow pathway” is not so much a pathway as a single chamber inside the mask, such as in a conventional face mask worn over the nose and mouth. The air inlet is the entire outer surface of the mask and the air is drawn into the chamber (flow pathway) where it is exposed to UV-C.

### EMBODIMENTS

Exemplary embodiments include the following:

A mask adapted to destroy pathogens prior to inhalation into the upper respiratory tract of a person, wherein, upon inhalation, air is drawn from the outside of the mask, through a flow pathway, to the inside of the mask, and thereby exposed to a germicidal a germicidal dose of UVC, wherein the mask comprises a mask body with an approximately hemispherical in shape, and is adapted to fit over the nose and mouth of a user, and wherein the mask comprises an outer surface and an inner surface, an air inlet penetrating from the outer surface to the inner surface, wherein the air inlet is flowably connected to the flow pathway having a first end, through which the air enters, and a second end, from which the air exits towards the mouth and nose of the user, whereby, upon inhalation, external atmospheric air flows through the inlet, through the first end of the flow pathway, through the length of the flow pathway, to the second end of the flow pathway, and exits the flow pathway towards the mouth and nose of the user, and wherein the mask further comprises a one-way valve positioned within the flow pathway that allows air to flow from the exterior to the interior of the mask, opening upon inhalation by the user (creating a negative pressure inside the mask), but closing upon exhalation of the user (creating a positive pressure inside the mask), and wherein the mask further comprises an LED UVC light source (one or more LEDs) with a wavelength of 200-280 nm, positioned within the mask, adapted to directly illuminate at least 85% of the total interior of the flow pathway, and wherein the UVC LED has a power rating from 0.5 mW to 12 W, and where the mask further comprises an exhalation vent, whereby exhaled air exits the mask, back into the atmosphere.

Other variations on this embodiment include:
having the exhalation vent comprising a valve positioned within the mask body.
wherein the flow pathway is at least 20, 30, 50, 60, 90 or 120 cm in total length.
the mask wherein the UVC LED has a power rating of 2 mW to 100 mW, or preferably 2 mW to 5 mW.
the mask wherein the UVC LED has power rating of 10 mW to 18 mW at 100 mA.
the mask wherein the UVC LED has power rating of 1 mW to 60 mW.
the mask wherein, when in use, the UVC LED provides an average power output over the interior surface area of the flow path is from 0.09 mW/cm2 to 24 mW/cm2.
the mask wherein, when in use, the UVC LED provides an average power output over the interior surface area of the flow path of from 0.1 mW/cm2 to 5 mW/cm2.
The mask wherein, when in use, the UVC LED provides an average power output over the interior surface area of the flow path of from 1 mW/cm2 to 4 mW/cm2.
The mask wherein the UVC LED has a total optical power output of 30 mW to 100 mW.
The mask wherein the UVC LED has a killing efficiency of >90% with a 20 second exposure.
The mask wherein the flow pathway comprises a number of approximately parallel pathways contiguously connected to each other and positioned in such a way with relation to the UVC LED such that at least 90% of the interior of the flow pathway is directly illuminated by UVC light.
The mask wherein the flow pathway comprises a number pathways arranged to radiate outwards from a central UVC source and positioned in such a way with relation to the UVC LED such that at least 90% of the interior of the flow pathway is directly illuminated by UVC light.
The mask wherein the flow pathway comprises two chambers, (1) an inhalation chamber proximal to the user, covering the user's nose and mouth, (2) a sterilizing anti-chamber, distal to the user, and a valve located between these two chambers, wherein the useable volume of the sterilizing anti-chamber is between 0.5 and 0.75 litres, and wherein the valve opens when the user breathes in, pulling a volume of air from the sterilizing anti-chamber into the inhalation chamber, and simultaneously pulling an equal volume of air from the outside into the sterilizing anti-chamber, wherein the UVC LED has a peak wavelength of 200 nm to 285 nm, and has a power rating of 2 mW to 3 mW.
The mask wherein the UVC LED has a power rating from 1 mW to 12 W.
The mask wherein the UVC LED produces a wavelength between 260 nm to 275 nm.
The mask with a power rating of 2 mW to 50 mW or preferably 100 mw to 1 W.
The mask wherein the UVC LED has a peak wavelength of 260 to 275 and a power of 2 mW to 3 mW.
The mask wherein the UVC LED has a killing efficiency of >90% with a 20 second exposure.
The mask wherein the UVC LED has a killing efficiency of >90% with a 20 second exposure
The mask wherein the UVC LED produces 2 mJ/cm2 of 222-nm light (inactivating >95% of aerosolized H1N1 influenza virus).

### GENERAL DISCLOSURES

This specification incorporates by reference all documents referred to herein and all documents filed concurrently with this specification or filed previously in connection with this application, including but not limited to such documents which are open to public inspection with this specification. As used in this specification, the singular forms “a”, “an”, and “the” include plural reference unless the context clearly dictates otherwise. Thus, for example, a reference to “a part” includes a plurality of such parts, and so forth.

The term “significant” is a semi-quantitate term that may mean at least 25% or 50% or 75% or 90% or 95% or 99% of an amount. For example, the air flow pathway is designed to maximize the residence time so that said particles receive a significant exposure to UV-C energy. In this case, a significant exposure may be an exposure that can on average or on a single application, kills or inactivates at least 50% of the organisms present in the volume exposed to the UV-C. Alternatively the percentage killed or inactivated could be 25% or 35% or 50% or 75% or 90% or 95% or 99%. Any range that including these amounts is also implied, for example from x % to y % or at least x % or between x % and y % or no more than x %, where x and y could be any number.

When “killing” of a pathogen or organism such as a bacteria or virus is mentioned, this includes and implies inactivation, attenuation, killing, denaturing, or otherwise affecting the pathogen so that infectivity or pathology is reduced. Organisms do not have to be killed in order to be changed in a way that reduces the significance or severity or amount of disease caused, and all these embodiments are explicitly included.

The “dwell time” or “residence time” refers to the time the pathogens are contained within the pathway, and therefore exposed to UV-C.

To say an LCD is rated from 1 mW to 12 W means that it consumes from 1 mW to 12 W of energy.

The term “comprises” and grammatical equivalents thereof are used in this specification to mean that, in addition to the features specifically identified, other features are optionally present. For example, a composition “comprising” (or “which comprises”) ingredients A, B and C can contain only ingredients A, B and C, or can contain not only ingredients A, B and C but also one or more other ingredients. The term “consisting essentially of” and grammatical equivalents thereof is used herein to mean that, in addition to the features specifically identified, other features may be present which do not materially alter the claimed invention.

The term “at least” followed by a number is used herein to denote the start of a range beginning with that number (which may be a range having an upper limit or no upper limit, depending on the variable being defined). For example, “at least 1” means 1 or more than 1, and “at least 80%” means 80% or more than 80%.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility).

When, in this specification, a range is given as “(a first number) to (a second number)” or “(a first number)-(a second number)”, this means a range whose lower limit is the first number and whose upper limit is the second number. For example, “from 40 to 70 nm” or “40-70 nm” means a range whose lower limit is 40 nm, and whose upper limit is 70 nm. When specific numbers are mentioned, it is implied that any range between these numbers may be used. For example if numbers 1, 5, 10 and 20 are mentioned, it is implied that ranges 1-20, 1-10, 1-5, 5-20, 5-20 etc. may also be used. Note that when various components are listed using the term “comprising”, the invention also implicitly encompasses combinations of compounds including other compounds, or lists of compounds excluding additional compounds.

All sets of amounts in this application, for example x and y, no matter what units are being used, include and imply ranges, for example from x % to y % or at least x % or between x % and y % or no more than x %, where x and y could be any number.

1. A mask adapted to destroy pathogens prior to inhalation into the upper respiratory tract of a person, wherein, upon inhalation, air is drawn from the outside of the mask, through a flow pathway, to the inside of the mask, and thereby exposed to a germicidal dose of UVC,
wherein the mask comprises a mask body with an approximately hemispherical in shape, and is adapted to fit over the nose and mouth of a user,
and wherein the mask comprises an outer surface and an inner surface,
an air inlet penetrating from the outer surface to the inner surface, wherein the air inlet is flowably connected to the flow pathway having a first end, through which the air enters, and a second end, from which the air exits towards the mouth and nose of the user, whereby, upon inhalation, external atmospheric air flows through the inlet, through the first end of the flow pathway, through the length of the flow pathway, to the second end of the flow pathway, and exits the flow pathway towards the mouth and nose of the user,
and wherein the mask further comprises a one-way valve positioned within the flow pathway that allows air to flow from the exterior to the interior of the mask, opening upon inhalation by the user (creating a negative pressure inside the mask), but closing upon exhalation of the user (creating a positive pressure inside the mask),
and wherein the mask further comprises one or more LED UVC light sources with a power rating from 0.5 mW to 12 W, each providing a wavelength of 200-280 nm, positioned within the mask, adapted to directly illuminate at least 85% of the total interior of the flow pathway,
And further comprising a power source electrically connected to the one or more LED UVC light sources,
and where the mask further comprises an exhalation vent, whereby exhaled air exits the mask, back into the atmosphere.

2. The mask of claim 1 wherein, in use or in a test situation, the total pressure drop across the mask is less than 0.05 w.c.

3. The mask of claim 2 wherein the flow pathway is at least 30 cm in total length.

4. The mask of claim 2 wherein the flow pathway is at least 60 cm in total length.

5. The mask of claim 4 wherein the one or more UVC LEDs has a power rating of 1 mW to 100 mW.

6. The mask of claim 5 wherein the one or more UVC LEDs has a total optical power output of from 10 mW to 100 mW.

7. The mask of claim 4 wherein the one or more UVC LEDs is powered by batteries with a total voltage of between 1.5V and 12V.

8. The mask of claim 3 wherein, when in use, the one or more UVC LEDs provides an average power output over the interior surface area of the flow path from 0.09 mW/cm² to 24 mW/cm².

9. The mask of claim 3 wherein, when in use, the one or more UVC LEDs provides an average power output over the interior surface area of the flow path from 1 mW/cm² to 4 mW/cm².

10. The mask of claim 3 having a killing efficiency of >90% when the pathogen is an influenza virus at 25 centigrade and 20% humidity.

11. The mask of claim 3 wherein the flow pathway comprises a number of approximately parallel pathways contiguously connected to each other and positioned in such a way with relation to the UVC LED such that at least 90% of the interior of the flow pathway is directly illuminated by UVC light.

12. The mask of claim 3 wherein the flow pathway comprises a number pathways arranged to radiate outwards from a central UVC source and positioned in such a way with relation to the UVC LED such that at least 90% of the interior of the flow pathway is directly illuminated by UVC light.

13. The mask of claim 2 wherein the flow pathway comprises two chambers, (1) an inhalation chamber proximal to the user, covering the user's nose and mouth, (2) a sterilizing anti-chamber, distal to the user, and a valve located between these two chambers, wherein the useable volume of the sterilizing anti-chamber is between 0.5 and 0.75 litres, and wherein the valve closes when the user breathes out and opens when the user breathes in, thereby pulling a volume of air from the sterilizing anti-chamber into the inhalation chamber, and simultaneously pulling an equal volume of air from the outside into the sterilizing anti-chamber, wherein the one or more UVC LEDs produces a peak wavelength between 200 nm to 280 nm, and wherein the UVC LED has a power rating of not more than 4 W.

14. The mask of claim 13 wherein the one or more UVC LEDs produce a peak wavelength of between 250 nm and 280 nm, with a total optical power output of from 10 mW to 100 mW.

15. The mask of claim 14 wherein the one or more UVC LEDs is powered by batteries having a total voltage of between 1.5V as 12V.

16. The mask of claim 14 wherein the sterilizing anti-chamber comprises on an inner surface, a reflective material that has a greater than 90% reflectivity for UVC.

17. The mask or claim 16 wherein the reflective material is e-PTFE.

18. The mask of claim 16 wherein the one or more UVC LEDs have the following specifications: forward current=500 mA; forward voltage=8.5V; radiant flux=70 mW; power dissipation=4.25 W; and Peak Wavelength=270-285 nm.

19. The mask of claim 16 having a killing efficiency of >90% when the pathogen is an influenza virus at 25 Centigrade and 20% humidity.

20. Than mask of claim 16 is rated with a total air resistance in use of from 0.01 to 0.25 inches w.c.
